(19) United States
(12) Patent Application Publication
(10) Pub. No.: US 2014/0363509 A1
(43) Pub. Date: Dec. 11, 2014

(54) COMPOSITIONS FOR THE TREATMENT OF MUCOUS MEMBRANE DISEASES

(71) Applicant: MISO S.r.I., Torino (IT)

(72) Inventor: Renato Silvio Mortera, Collegno (Torino) (IT)

(73) Assignee: MISO S.r.I., Torino (IT)

(21) Appl. No.: 14/369,558

(22) PCT Filed: Nov. 27, 2012
(86) PCT No.: PCT/IB2012/056763

§ 371 Date: Jun. 27, 2014

(30) Foreign Application Priority Data
Dec. 30, 2011 (IT) TO2011A001242

(51) Int. Cl. A61K 9/14; A61K 31/4164; A61K 47/06; A61K 31/7056

(52) U.S. Cl. CPC A61K 9/143; A61K 31/7056; A61K 31/4164; A61K 47/06
USPC 424/489; 514/23; 514/396

(57) ABSTRACT

Composition comprising at least one active principle, at least one vehicle for said active principle and particles of porous silica, in which the active principle is contained in at least one pore of at least a first portion of such silica particles and in that vehicle, for use in the treatment of a disease of a mucous membrane.

### FIELD OF THE INVENTION

The present disclosure relates to compositions for topical application to mucous membranes.

### TECHNOLOGICAL BACKGROUND

There are several examples of products for topical treatment of mucous membranes. The term “mucus” is generally used to indicate the heterogeneous secretions that cover epithelial cells (Marriot, C. and Gregory, N P, 1990. Mucus physiology and pathology. In: V. Lenaerts and R. Gurny (Eds), Bioadhesive Drug Delivery Systems, CRC Press, Boca Raton, pp. 1-24). Mucus is produced in many parts of the body, including the ear, eye, nose, mouth, and the gastrointestinal reproductive and respiratory tracts. Its main role is to protect and lubricate the underlying epithelial tissue.

Of particular interest in this context are products based on mucoadhesion, i.e., the adhesion of a natural or synthetic polymer to a biological substrate. These represent a practical method for the immobilization or positioning of drugs and an important new aspect of their controlled release. Although the concept of mucoadhesion is not new, in recent years there has been a sharp increase in interest in the use of mucoadhesive polymers for drug delivery (Marriot, C. and Gregory, N P, 1990. Mucus physiology and pathology. In: V. Lenaerts and R. Gurny (Eds), Bioadhesive Drug Delivery Systems, CRC Press, Boca Raton, pp. 1-24. Marriot, C. and Hughes, D. R. L., 1990, Mucus physiology and pathology. In: R. Gurny and H. E. Junginger (Eds), Bioadhesion-Possibilities and Future Trends, Wissenschaftliche Verlagsgesellschaft mbH, Stuttgart, pp. 29-43). For this reason possible changes to existing bioadhesive materials are increasingly gaining significance, as is the diffusion of various drugs from bioadhesive devices.

In this context, formulations capable of providing a constant concentration of active principle at the site of application, both in mucoadhesive devices and in conventional preparations, for a prolonged time, thus reducing the number of applications, represent a significant improvement of the products now on the market.

Such formulations may be obtained by using specific materials as carriers of the active principle, whose physical characteristics allow a controlled release of the same.

Mesoporous materials and in particular mesoporous silicas were proposed for the first time as vehicles for the controlled release of drugs in 2001 by Vallet-Regi et al. (Chem. Mater., 2001; 13:308).

Such materials have pores that are characterized by a high specific surface area, greater than 1000 m²/g (C. T. Kresge et al., Nature, 1992; 359:710) and by a diameter that can be modulated by varying the synthesis conditions.

Because of these characteristics, mesoporous materials and in particular mesoporous silicas, for example in the form of particles, are used to incorporate, carry and release active principles in a variety of substrates.

For example, the international patent application WO-A-2005/009602 describes particles of mesoporous silica made to carry substances such as drugs, polynucleotides, polypeptides, hormones, enzymes, which are loaded inside the pores of the particles. In addition, these particles have plugs that function to close the pores to slow or prevent the release of the encapsulated agent.

The Japanese patent application JP-A-2009013142 describes mesoporous silicas made to contain and carry active principles across cell membranes.

The international patent application WO-A-2009/110939 describes useful devices for the transport and release of bioactive agents, such as drugs, in combination with radioisotopes for the chemo-, radio-therapeutic treatment of tumours. Such devices may be represented by particles of mesoporous silica.

Devices comprising mesoporous materials provide drug release, but do not resolve the issues of programmed or prolonged release.

### SUMMARY OF THE INVENTION

Bearing these premises in mind, the need is felt for more effective, improving solutions that provide a composition for use in the treatment of diseases of mucous membranes capable of constant and prolonged releasing of at least one active principle.

According to the invention, such object is achieved by means of the solution recalled specifically in the annexed claims, which form an integral part of the present description.

The present description relates to a composition comprising at least one active principle located both inside of porous silica particles and dispersed within the composition, where the composition is intended for the treatment of a disease of a mucus membrane.

In one embodiment, the composition comprises at least one active principle, at least one vehicle for the active principle and particles of porous silica, wherein the active principle is contained in at least one pore of a portion of such silica particles and in said vehicle for use in the treatment of a disease of a mucous membrane.

A second embodiment of the present description relates to a composition that comprises an active principle, a vehicle for the active principle and particles of porous silica, wherein the active principle is contained in at least one pore of at least a portion of such silica particles and in said vehicle, the composition comprising also at least a further active principle, at least one additional vehicle for such further active principle, the further active principle being contained in at least one pore of a second portion of the silica particles and in the at least one further vehicle, and the further active principle being soluble in such further vehicle for use in the treatment of a disease of a mucous membrane.

### DETAILED DESCRIPTION OF EMBODIMENTS

The invention will now be described in detail, by way of non-limiting example, with reference to the realization of a composition for the controlled release of an active principle for use in the treatment of a disease of a mucous membrane.

In the following description, numerous specific details are presented to provide a thorough understanding of the embodiments. The embodiments may be practiced without one or more of the specific details, or with other methods, components, materials, etc. In other cases, well known structures, materials or operations are not shown or described in detail to avoid obscuring certain aspects of the embodiments.

Throughout the present description, reference to “one embodiment” or “embodiment” means that a particular configuration, structure, or characteristic described. in connection with the embodiment is included in at least one embodiment. Thus, the appearance of the phrases “in one embodiment” or “in a certain embodiment” in various places throughout the present description do not necessarily refer to the same embodiment. Furthermore, the particular configurations, structures, or characteristics may be combined in any suitable manner in one or more embodiments.

The headings used herein serve simply for convenience and do not interpret the scope or meaning of the embodiments.

Examples of treatments that may benefit from a composition object of the present description capable of controlled and prolonged release, possibly in combination with mucoadhesive systems, are topical therapies for diseases of the genital tract, preferably vaginal.

Among the most common diseases it is possible to cite the bacterial vaginoses currently treated with GYNOCANESTEN, a topical antimycotic belonging to the group of imidazole derivatives, a product from Bayer that contains clotrimazole. In this context, double-sided adhesive systems to be applied directly on the vaginal mucosa have been developed, such as for example Clindesse™ (http://www.clindesse.com). This technology allows a formulation of 2% clindamycin phosphate to adhere to the walls of the vagina and to remain where it is needed, in order to effectively and locally treat the bacteria responsible for the infection.

However, this system does not provide a particularly effective treatment as the active principle is not released gradually over a period of time that is long enough to reduce the number of applications required.

The composition object of the present description provides, for example, a formulation of 2% clindamycin phosphate in contact with the mucous membrane with a prolonged action of at least 72 hours, even in the absence of a mucoadhesive device. Thus, with a single application, this provides a dosage that would require three applications of the product currently available commercially.

Another area of interest is nasal release: the nasal cavity performs important protective functions in the respiratory system in that it filters, warms and humidifies the inhaled air. Inhaled droplets or particles are trapped by the hairs in the nasal vestibule or by the mucous layer in the main cavity, which gradually carries them to the back of the throat and down into the gastrointestinal tract.

The nasal mucosa is the ideal place for the application of compositions of the present description, possibly in combination with bioadhesive active principle release systems, for the treatment of various diseases, such as allergic rhinitis, and nasal polyps. Polyposis is one of the diseases for which various medical therapies can be used, unfortunately with results that are often inadequate and only temporary. Therapies normally used are (i) nasal sprays with topical steroids; (ii) nasal sprays based on distilled and sterilized sea water; (iii) oral or parenteral corticosteroids; (iv) antihistamines, (v) leukotriene inhibitors; (vi) products for topical use based on antagonists of the HMGB1 protein and antiedemic agents (mannitol and albumin).

The bioavailability and the retention time of nasally administered active principle(s) may be increased by employing the composition object of the present description, possibly in combination with bioadhesive release systems.

Finally, a third example of a topical therapy of a mucous membrane is therapy on the buccal mucosa in the field of dentistry.

An embodiment of the present description concerns a composition comprising:
at least one active principle,
at least one vehicle for said active principle,
particles of porous silica,
   wherein said active principle is contained in at least one pore of at least a first portion of said silica particles, and in said vehicle for use in the treatment of a disease of a mucous membrane.

The active principle is contained in the vehicle at a concentration equal to its saturation concentration in such vehicle. In the case in which the composition contains a second vehicle, such second vehicle will be chosen so as not to solubilise the active principle contained in the silica particles.

A second embodiment of the present description concerns a composition that comprises an active principle, a vehicle for the active principle and porous silica particles, wherein the active principle is contained in at least one pore of at least a portion of such silica particles and in said vehicle, the composition comprising also at least a further active principle, at least one additional vehicle for such further active principle, the further active principle being contained in at least one pore of a second portion of the silica particles and in the at least one further vehicle, and the further active principle being soluble in such further vehicle for use in the treatment of a disease of a mucous membrane.

The further active principle is contained in the further vehicle for such further active principle at a concentration equal to its saturation concentration in that further vehicle.

The further active principle is insoluble or sparingly soluble in the vehicle in which the first active principle is dissolved and—in parallel—the first active principle is insoluble or sparingly soluble in the further vehicle in which the further active principle is dissolved.

The vehicle(s) used in the compositions object of the present description may be hydrophilic vehicle(s) or lipophilic vehicle(s) and are selected as a function of the solubility of the active principle (s) used in the respective vehicle.

Among the hydrophilic vehicles advantageously usable in the present compositions, water and alcohols can be mentioned by way of non-limiting example.

Among the lipophilic vehicles advantageously usable in the present compositions, Vaseline oil, paraffin oil, castor oil can be mentioned by way of non-limiting example.

The compositions object of the present description are capable of maintaining a therapeutic action that is prolonged for several days at the site of application, optimizing the time of treatment and avoiding the numerous and repeated applications typical of the current treatments.

Furthermore, the silica particles act as protection against the active principles themselves by limiting direct and harmful contact between the loaded drugs and mucous membranes and avoiding possible irritation or allergies.

By increasing or decreasing the amount of silica particles present in the composition it is possible to obtain a constant release of the active principle(s) for a period of time comprised between 1 and 10 days.

The compositions object of the present description may be destined for the treatment of diseases such as, by way of non-limiting example, mucosal bacterial vaginosis, mycotic vulvovaginitis, non-infectious vulvovaginitis, trichomoniasis, scleroatrophy, canker sores, herpes, stomatitis, lichen planus, allergic rhinitis, polyps, abscesses, fistulas, ano-rectal disorders.

The compositions object of the present description may be used on the mucous membrane of the reproductive system, intestine, oral and nasal cavities.

Preparing mesoporous silica in the form of sub-micrometer particles of controlled morphology provides finely divided powders that can be dispersed in preparations such as creams, ointments, gels, pomade, lotions and foams while maintaining the desired properties, such as viscosity and consistency, for topical application on mucous membranes.

Classes of active principles that can be used are, for example, antibiotics, antifungals, anti-inflammatories, antiseptics, analgesics, steroids, antivirals, calcineurin inhibitors, retinoids, antihistamines, anticholinergics, decongestants, cicatrizants.

Unexpectedly, it was verified that, despite the presence of silica particles, the compositions object of the present invention may be used for the treatment of mucous membranes, known to be very sensitive because of their high degree of innervation. It was verified that the composition described herein does not cause irritating effects on the mucous membranes on which it is applied.

The technology developed for the release of drugs by mesoporous silicas makes it possible to incorporate a quantity of active principle (AP) within particles of porous silica, preferably silica particles having sub-micrometric dimensions and still more preferably sub-micrometer silica particles with controlled porosity. Such particles can be prepared by following various procedures described in the literature (M. Grün et al. Microporous Mesoporous Mat. 27 (1999) 207; C Y Lai et al., J. Am. Chem. Soc. 125 (2003) 4451; Y. Yamada and K. Yano, Microporous Mesoporous Mat. 93 (2006) 190) that envision the use of a surfactant as templating agent (usually hexadecyltrimethylammonium bromide (C₁₆TMABr)) dissolved in a basic water-ethanol-ammonia solution and a silica source (usually tetraethylorthosilicate (TEOS)). They have cylindrical pores with hexagonal symmetry and a monodisperse distribution of diameters varying from 2 to 10 nm, depending on synthesis conditions.

Mesoporous silicas are currently sold by Sigma-Aldrich and ACS Material (http://www.acsmaterial.com).

Employing sub-micrometric silica particles with controlled porosity it is possible to vary the amount of active principle loaded, comprised between 10% and 50% by weight of the particles, depending on the porosity chosen and the on the AP itself, as long as the AP does not exceed the maximum dimensions obtainable for the pores of the silica.

Loading of the active principle into the pores of the mesoporous silica particles is carried out by contacting such particles with a solution containing the AP dissolved at a known concentration. Ethanol, methanol, pentane, hexane, acetone, water and buffer solutions are among the solvents most used for the loading of AP in mesoporous silicas.

A possible alternative to incorporation from solutions is the use of supercritical CO₂ as a solvent medium.

The compositions object of the present description can be used directly on sites of interest or combined with mucoadhesive products to prolong the efficacy.

The compositions object of the present description may be administered in the form of a cream, pomade, ointment, paste or gel, or be contained in a mucoadhesive system.

Indications will now be provided for the realization of a composition comprising a single active principle. However, it is clear that these indications are examples, it being possible to realize—according to the indications provided herein—compositions containing more than one active principle.

In a particular embodiment, the particles containing the AP are dispersed in a eudermic composition comprising at least two vehicles:
a first vehicle, in which the active principle is soluble (referred to as vehicle A), is chosen from among water or an alcohol or a mixture of these, and
a second vehicle, in which the active substance is not soluble or is sparingly soluble (referred to as vehicle B), chosen from among Vaseline oil, castor oil, or paraffin oil.

In this way it is possible to control the concentration of active principle present in the composition by acting exclusively on the amount of active principle dissolved in vehicle A (M_{AP}).

In particular, if the concentration of AP in the vehicle A (C_{AP,v.A}) is maintained equal to its saturation concentration or solubility (C_{S,AP-A}), it is possible to modify the concentration in the composition (C_{AP,c.}) by simply varying the volume of the vehicle A (V_{A}) compared to vehicle B (V_{B}), as described by the following equations:

C_{AP,v.A}=M_{AP}/V_{A}=C_{S,AP-A} (1)

C_{AP,c.e}=M_{AP}/(V_{A}+V_{B}) (2)

from which:

C_{AP,c.e.}=V_{A}*C_{S,AP-A}/(V_{A}+V_{B}) (3)

Dispersing the silica particles containing the AP in the composition prepared at the desired C_{AP,c.}, they act as a reservoir of AP, maintaining the concentration of AP in the vehicle A (C_{AP,v.A}) equal to its saturation concentration (solubility) until the pores are completely empty.

In this way it is possible to maintain the AP concentration in the composition constant for a time that can be modulated as needed by increasing or decreasing the amount of silica particles containing the AP.

### EXAMPLE 1

### Clindamycin-Based Vaginal Cream for the Treatment of Bacterial Vaginosis (Clindamycin Phosphate 2.376%)

2.376 g of clindamycin phosphate (CP) are added to 97.624 g of the cream made from a vehicle A (liquid paraffin, in which CP is not soluble) in the amount of 50.1 g and a vehicle B (water) in the amount of 47.6 g.

Then, to 100 g of cream a certain amount of sub-micrometric silica particles with 2 nm diameter pores containing 20% CP by weight are added, obtained by contacting the silica particles with an aqueous CP solution with agitation at room temperature for 4 hours.

1a—When the amount of particles added to the preparation is 15.8 g, a cream is obtained that ensures a constant release of CP for 48 hours, and that requires a single application (5 g) every two days, instead of the daily application now required for preparations of this type that are currently on the market.

1b—Increasing the amount of particles to 23.76 g per 100 g of cream assures constant release for 72 hours, making a single application necessary every three days.

### EXAMPLE 2

### Clotrimazole-Based (2%) Vaginal Cream for the Topical Treatment of Vaginal Candidiasis

2 g of Clotrimazole are added to 98 g of cream comprising a vehicle A (liquid paraffin) and a vehicle B (water, in which the AP is not soluble) in proportions such that the Clotrimazole is at its saturation concentration.

Then, to 100 g of cream a certain amount of sub-micrometric silica particles with 2 nm diameter pores, containing 20% Clotrimazole by weight are added, obtained by contacting the silica particles with a solution of Clotrimazole in acetone, with agitation at room temperature for 4 hours.

2a—When the amount of particles added to the preparation is 13.3 g, a cream is obtained that ensures a constant release of Clotrimazole for 48 hours, and that requires a single (5 g) application every two days, instead of the daily applications now required for preparations of this type that are currently on the market.
2b—Increasing the amount of particles to 20 g per 100 g of cream assures constant release for 72 hours, making a single application necessary every three days.

The presence of the silica particles does not alter the form or consistency of a preparation currently on the market, such as GYNOCANESTEN.

Naturally, while the principles of the invention remain constant, the structural details and the embodiments may vary widely with reference to what has been described and illustrated by way of example only, without departing from the scope of the present invention.

1. Composition comprising:
at least one active principle,
at least one vehicle for said active principle,
porous silica particles,
wherein said active principle is contained in at least one pore of at least a first portion of said silica particles, and in said vehicle, for use in the treatment of a disease of a mucous membrane.

2. Composition according to claim 1, comprising at least a further vehicle.

3. Composition according to claim 1, wherein the porous silica particles are silica particles having sub-micrometer size, and wherein the pores of said silica particles have a size comprised in the range 2 nm to 50 nra.

4. Composition according to claim 1, wherein said active principle is contained in said vehicle at a concentration equal to its saturation concentration in said vehicle.

5. Composition according to claim 1, wherein said active principle contained in said first portion of said porous silica particles is present in an amount comprised in the range 10% to 50% by weight of said portion.

6. Composition according to claim 2, wherein said active principle is insoluble or poorly soluble in said at least one further vehicle.

7. Composition according to claim 2, wherein said composition comprises, in addition, at least a further active principle, said further active principle being contained in at least one pore of a second portion of said silica particles and in said at least one further vehicle, the active principle being soluble in said at least one further vehicle.

8. Composition according to claim 7, wherein said further active principle is contained in said at least one further vehicle at a concentration equal to its saturation concentration in said at least one further vehicle.

9. Composition according to claim 2, wherein said further active principle is insoluble or poorly soluble in said vehicle for said active principle.

10. Composition according to claim 1, wherein said vehicle and said at least one further vehicle are a hydrophilic vehicle and/or a lipophilic vehicle.

11. Composition according to claim 1, wherein said active principle and/or said further active principle is released at a constant concentration to said mucous membrane for a period of time between 1 and 10 days.

12. Composition according to claim 1, wherein said active principle and/or said further active principle is/are pharmacologically active principle (s).

13. Composition according to claim 1, wherein said disease is selected from: mucosal bacterial vaginosis, mycotic vulvovaginitis, non-infectious vulvovaginitis, trichomoniasis, scleroatrophy, canker sores, herpes, stomatitis, lichen planus, allergic rhinitis, polyps, abscesses, fistulas, ano-rectal disorders.

14. Composition according to claim 1, wherein said mucous membrane is selected from: genital, nasal, oral and intestinal.

15. Composition according to claim 1, wherein said active principle is selected from: antibiotics, antimycotic agents, antiinflammatory agents, antiseptics, analgesics, steroids, antiviral agents, calcineurin inhibitors, retinoids, antihistamines, anticholinergic agents, decongestants, healing agents.

16. Composition according to claim 1, wherein said composition is in the form of cream, ointment, pomade, gel, paste, lotion, foam.

17. Composition according to claim 1, wherein said composition is contained in a mucoadhesive system.
